(19) United States
(12) Patent Application Publication
(10) Pub. No.: US 2018/0207068 A1
(43) Pub. Date: Jul. 26, 2018

(54) COSMETIC AGENTS CONTAINING A COMBINATION OF AT LEAST TWO DIFFERENT ACTIVE INGREDIENTS

(71) Applicant: Henkel AG & Co. KGaA, Duesseldorf (DE)

(72) Inventor: Thomas Doering, Duesseldorf (DE)

(73) Assignee: Henkel AG & Co. KGaA, Duesseldorf (DE)

(21) Appl. No.: 15/876,228

(22) Filed: Jan. 22, 2018

(30) Foreign Application Priority Data
Jan. 23, 2017 (DE) 102017201025.8

(51) Int. Cl. A61K 8/36; A61K 8/34; A61K 8/37; A61Q 15/00

(52) U.S. Cl. CPC A61K 8/36; A61K 8/345; A61Q 15/00; A61K 8/34; A61K 8/37

(57) ABSTRACT

Cosmetic agents containing a combination of at least two different active ingredients are provided. In one example, a cosmetic agent, in a cosmetically compatible carrier, includes 2-butyloctanoic acid and at least one diol of the formula (I)

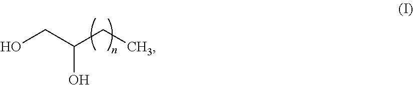

wherein n denotes an integer selected from the group of 3, 5, 7 and 9. The total amount of compound(s) of the formula (I) is from about 0.05 to about 10 wt. %, relative to the agent.

### CROSS-REFERENCE TO RELATED APPLICATION

This application claims priority to German Patent Application No. 10 2017 201 025.8, filed Jan. 23, 2017, which is incorporated herein by reference in its entirety.

### TECHNICAL FIELD

The present application relates to cosmetic agents which contain a combination of at least two active ingredients. These cosmetic agents have an outstanding deodorizing effect which also lasts for a long time.

### BACKGROUND

Furthermore, the present disclosure relates to a method for reduction and/or prevention of the body odor released by perspiration, wherein an inventive cosmetic agent is applied to the skin and remains in place for at least one hour.

Finally, the present disclosure relates to the use of an inventive cosmetic agent for reduction and/or prevention of the body odor released by perspiration.

Eccrine and apocrine sweat glands are present in the human armpit. Whereas the eccrine glands produce an aqueous secretion in response to heat, the apocrine glands can secrete a viscous secretion in response to stress. This apocrine sweat constitutes a complex mixture containing, among other constituents, steroids, cholesterol and other fats, as well as approx. 10% of proteins. The bacterial decomposition of the constituents of the apocrine sweat causes the secretion, which is initially odorless, to produce an unpleasant body odor under the armpit.

The decomposition of the apocrine sweat, which contributes substantially to body odor, more particularly to axillary body odor, can be divided into three classes: the first class comprises short-chained C4-C10 fatty acids, which can be linear, branched, saturated and unsaturated (for example, isovaleric acid, 3-methyl-2-hexenoic acid, 3-hydroxy-3-methylhexanoic acid), the second class comprises short-chained, linear or branched sulfonyl alcohols, the third class comprises various steroid hormones and the metabolic products thereof (for example, 5-α-androstenol and 5-α-androstenone).

Accordingly, body odor can be combated by preventing the bacterial decomposition of the sweat or by using perfume to conceal the body odor. In the prior art, the bacterial decomposition of sweat is prevented through the use of antimicrobial substances, which reduce the number of sweat-decomposing bacteria on the skin by employing elimination, or inhibit the growth of said bacteria. Furthermore, known active ingredients reduce and/or prevent the formation of decomposition products by blocking bacterial enzymes. A method of absorbing the volatile decomposition products by employing physical and/or chemical interaction and thus preventing unpleasant body odor is also known. Moreover, the formation of body sweat can be prevented with the use of antiperspirant active ingredients so that there is less sweat-decomposing bacteria and/or no decomposition products are formed. However, there is still a need for cosmetic agents that have a high and long-lasting deodorizing effect.

Use of 2-butyloctanoic acid as a deodorizing agent is known in the prior art. European Patent EP 0 742 004 B1 discloses the use of dialkyl-substituted acetic acids such as 2-butyloctanoic acid as antibacterial, antimycotic or antiviral active ingredients.

Active ingredient combinations of dialkyl-substituted acetic acids with one or multiple substances selected from the group of monoglycerol monoalkyl ethers, diglycerol monoalkyl ethers, triglycerol monoalkyl ethers, monoglycerol dialkyl ethers, diglycerol dialkyl ethers and triglycerol dialkyl ethers are disclosed in EP 1 461 004 B1.

### BRIEF SUMMARY

Cosmetic agents and methods including cosmetic agents are provided herein. In an exemplary embodiment, a cosmetic agent comprises, in a cosmetically compatible carrier,

a) 2-butyloctanoic acid and

b) at least one diol of the formula (I)

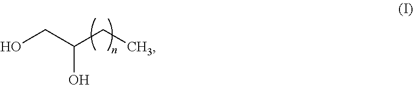

wherein n denotes an integer selected from the group of 3, 5, 7 and 9, wherein the total amount of compound(s) of the formula (I) is from about 0.05 to about 10 wt. %, relative to the total weight of the cosmetic agent.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The objective of the present disclosure is to prepare cosmetic agents which achieve a good and long-lasting effect against body odor. Furthermore, the occurrence of skin irritations during and/or after use of the inventive cosmetic agent should be avoided. The cosmetic agents should also have a high storage stability and good cosmetic properties.

Surprisingly, it has now been found that the use of a combination of 2-butyloctanoic acid having one or multiple diols does not specifically combat the bacteria causing odor, rather an unexpectedly long deodorizing effect is achieved. With the synergistic effect of the two aforementioned components of the inventive cosmetic agent, the amount of active ingredients can be reduced without negatively influencing the deodorizing effect. Due to the reduced amount of active ingredients, the inventive cosmetic agents have a low irritating and sensitizing effect. Furthermore, the use of the active ingredient combination does not have a negative influence on the storage stability of the inventive cosmetic agents so that said agents have excellent storage stability. Finally, these agents have good cosmetic properties.

A first subject matter of the present disclosure is therefore a cosmetic agent containing, in a cosmetically compatible carrier,

a) 2-butyloctanoic acid and
b) at least one diol of the formula (I)

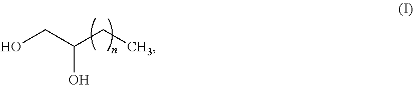

wherein n denotes an integer from the group 3, 5, 7 or 9
with the condition that the total amount of compound(s) of the formula (I) is from about 0.05 to about 10 wt. %, relative to the agent.

With the use of a combination of 2 butyloctanoic acid with at least one diol of the formula (I), an outstanding reduction of the body odor released by perspiration, particular body odor under the arm, is achieved. Furthermore, it has been found that the aforementioned combination of the two components a) and b) of the inventive cosmetic agents has a synergistic increase of the deodorizing benefit so that the amount of active ingredients can be reduced without negatively influencing the deodorizing effect. The inventive cosmetic agents have improved skin compatibility due to the reduced amount of active ingredients. In addition, use of the aforementioned combination of active ingredients can improve the cosmetic properties of the inventive cosmetic agents. Finally, use of the components a) and b) does not have a negative influence on the storage stability of the inventive cosmetic agent.

Unless otherwise specified, the wt. % presently relates to the total weight of the inventive cosmetic agents, wherein the sum of all ingredients of the inventive agents amounts to 100 wt. %. Moreover, unless otherwise specified, the wt. % relates to the amount of the respective component in the propellant-free cosmetic agent so that the amount of propellant, if present, is not taken into account with the calculation of the total weight of cosmetic agents.

The cosmetic agent contains the components a) and b) in a cosmetically compatible carrier. Said carrier preferably contains at least one component, selected from water, a C1-C4-alcohol, a cosmetic oil that is liquid under normal conditions, as well as mixtures thereof. The cosmetic oils that are liquid under normal conditions are not miscible with water and are neither aromatic substances nor essential oils. In the context of the present application, “normal conditions” constitute a temperature of 20° C. and a pressure of 1,013 hPa.

Suitable cosmetic carriers are, for example, aqueous or aqueous-alcoholic carriers. Carriers in this context are understood to mean carriers which contain more than 5.0 wt. % water or more than 5.0 wt. % water and at least one C1-C4-alcohol, relative to the total weight of the cosmetic agent in each case. Aqueous carriers preferably contain free water in a total quantity of from about 10 to about 96 wt. %, preferably from about 15 to about 80 wt. %, more preferably from about 30 to about 70 wt. %, particularly from about 40 to about 60 wt. %, relative to the total weight of the cosmetic agent. Aqueous-alcoholic carriers preferably contain ethanol in a total quantity of from about 0.1 to about 50 wt. %, preferably from about 0.5 to about 30 wt. %, more preferably from about 1.0 to about 20 wt. %, particularly from about 1.0 to about 9.0 wt. %, relative to the total weight of the cosmetic agent.

The aforementioned aqueous and aqueous-alcoholic carriers can also contain at least one (C2-C6)-alkyl alcohol having 2 or 3 hydroxy groups, particularly 1,2-propylene glycol, 1,3-propylene glycol, glycerin and 1,3-butylene glycol.

However, use of an anhydrous cosmetic carrier can also be stipulated. As contemplated herein, anhydrous carriers are those containing less than 5.0 wt. %, preferably less than 4.0 wt. %, more preferably less than 3.0 wt. %, most preferably 0 wt. %, relative to the total weight of the cosmetic agent, of free water. Free water within the context of the present disclosure is understood to mean water that is different from crystal water, hydration water or similar molecularly bound water of the components that are used. However, water of crystallization, water of hydration or water with similar molecular bonds of the used components are not taken into account in the calculation of the total amount of free water.

In this context, alcoholic media, in particular, are used as cosmetically compatible carriers. Low alcohols having from about 1 to about 4 carbon atoms are normally used for cosmetic purpose, such as ethanol and isopropanol, can be used, in particular as alcohols. Alcoholic carriers preferably contain ethanol in a total quantity from about 20 to about 95 wt. %, preferably from about 25 to about 65 wt. %, particularly from about 25 to about 60 wt. %, relative to the total weight of the cosmetic agent.

Furthermore, use of liquid cosmetic oils as cosmetic carriers is preferable in this context. These cosmetic oils can be selected from the group of (i) volatile non-silicone oils, particularly liquid paraffin oils and isoparaffin oils, such as isodecane, isoundecane, isododecane, isotridecane, isotetradecane, isopentadecane, isohexadecane and isoeicosane; (ii) non-volatile non-silicone oils, in particular the esters of linear or branched saturated or unsaturated C2-30-fatty alcohols with linear or branched saturated or unsaturated C2-30-fatty acids which can be hydroxylated, the C8-C22-fatty alcohol esters of monohydric or polyhydric C2-C7-hydroxycarboxylic acids, the dicarboxylic acid esters of linear or branched C2-C10-alkanols, the addition products of ethylene oxide and/or propylene oxide onto mono- or multivalent C3.22-alkanols, which may optionally be esterified, the symmetrical, asymmetric or cyclic esters of carbonic acid with fatty alcohols, the esters of dimeric unsaturated C12-22-fatty acids with monohydric, linear, branched and cyclic C2-18-alkanols or C2-6-alkanols, the benzoic esters of linear or branched C8-22-alkanols, such as the benzoic acid C12-15-alkyl ester and the benzoic acid isostearyl ester and benzoic acid octyldodecyl ester, the synthetic hydrocarbons, such as polyisobutene and polydecenes, the alicyclic hydrocarbons; and (iii) mixtures thereof.

As contemplated herein, the expression “liquid cosmetic oil” refers to cosmetic oils which have a vapor pressure of from about 2.66 Pa to about 40,000 Pa (from about 0.02 to about 300 mm Hg), preferably from about 10 to about 12,000 Pa (from about 0.1 to about 90 mm Hg), more preferably from about 13 to about 3,000 Pa (from about 0.1 to about 23 mm Hg), most preferably from about 15 to about 500 Pa (from about 0.1 to about 4 mm Hg) at 20° C. and an environmental pressure of 1,013 hPa. Moreover, the expression “non-volatile cosmetic oils” as contemplated herein means oils which have a vapor pressure of less than 2.66 Pa (0.02 mm Hg) at 20° C. and an environmental pressure of 1,013 hPa.

As contemplated herein, the preference is for the use of mixtures of the aforementioned cosmetic oils, particularly non-volatile and volatile cosmetic oils, since this enables parameters such as skin feel, visibility of residue and stability of the cosmetic agent as contemplated herein to be adjusted, thereby allowing the agent to be better adapted to the needs of the consumer.

According to the present disclosure, the cosmetic oil, which is liquid at 20° C. and 1,013 hPa, is ideally contained in a total quantity of from about 1.0 to about 98 wt. %, preferably from about 2.0 to about 95 wt. %, more preferably from about 5.0 to about 70 wt. %, even more preferably from about 10 to about 60 wt. %, particularly from about 15 to about 50 wt. %, relative to the total weight of the cosmetic agent.

The inventive cosmetic agent contains 2-butyloctanoic acid as a first essential component a):

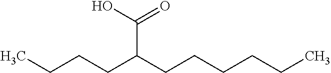

This is preferably used within a specific quantity range. Preferred agents as contemplated herein are exemplified in that they contain from about 0.01 to about 1 wt. %, preferably from about 0.02 to about 0.8 wt. %, more preferably from about 0.03 to about 0.7 wt. %, even more preferably from about 0.04 to about 0.6 wt. % and particularly from about 0.05 to about 0.5 wt. % 2-butyloctanoic acid, relative to their weight.

As a second essential constituent b), the cosmetic agent as contemplated herein contains at least one diol of the Formula (I).

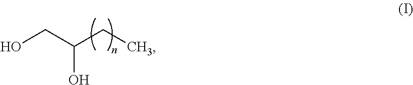

wherein n denotes an integer from the group 3, 5, 7 or 9, with the condition that the total amount of compound(s) of the formula (I) is from about 0.05 to about 10 wt. %, relative to the agent. Therefore, inventive cosmetic agents contain, relative to their weight from about 0.05 to about 10 wt. %
from about 0.05 to about 10 wt. % 1,2-hexanediol (n=3) or
from about 0.05 to about 10 wt. % 1,2-octanediol n=5) or
from about 0.05 to about 10 wt. % 1,2-decanediol (n=7) or
from about 0.05 to about 10 wt. % 1,2-dodecanediol (n=9) or
from about 0.05 to about 10 wt. % of a mixture of 1,2-hexanediol (n=3) and 1,2-octanediol (n=5) or
from about 0.05 to about 10 wt. % of a mixture of 1,2-hexanediol (n=3) and 1,2-decanediol (n=7) or
from about 0.05 to about 10 wt. % of a mixture of 1,2-hexanediol (n=3) and 1,2-dodecanediol (n=9) or
from about 0.05 to about 10 wt. % of a mixture of 1,2-octanediol (n=5) and 1,2-decanediol (n=7) or
from about 0.05 to about 10 wt. % of a mixture of 1,2-octanediol (n=5) and 1,2-dodecanediol (n=9) or
from about 0.05 to about 10 wt. % of a mixture of 1,2-decanediol (n=7) and 1,2-dodecanediol (n=9) or
from about 0.05 to about 10 wt. % of a mixture of 1,2-hexanediol (n=3) and 1,2-octanediol (n=5) and 1,2-decanediol (n=7) or
from about 0.05 to about 10 wt. % of a mixture of 1,2-hexanediol (n=3) and 1,2-octanediol (n=5) and 1,2-dodecanediol (n=9) or
from about 0.05 to about 10 wt. % of a mixture of 1,2-hexanediol (n=3) and 1,2-decanediol (n=7) and 1,2-dodecanediol (n=9) or
from about 0.05 to about 10 wt. % of a mixture of 1,2-octanediol (n=5) and 1,2-decanediol (n=7) and 1,2-dodecanediol (n=9) or
from about 0.05 to about 10 wt. % of a mixture of 1,2-hexanediol and 1,2-octanediol and 1,2-decanediol and 1,2-dodecanediol.

Particular preference is given to the compound(s) of the formula (I) used in specific quantities. The inventive agents have proven to be particularly effective as a deodorant if they contain the compound(s) of the formula (I) in quantities from about 0.1 to about 5.0 wt. %, preferably from about 0.15 to about 2.5 wt. %, more preferably from about 0.175 to about 1.0 wt. %, even more preferably from about 0.2 to about 0.8 wt. % and particularly from about 0.25 to about 0.7 wt. %, relative to the total propellant-free agent in each case.

Among the compounds of formula (I), the two representatives with n=3 and n=5 have been found to be especially effective. Therefore, preference is given to inventive agents which contain at least one compound of the group 1,2-hexanediol, 1,2-octanediol. In summary, particular preference is given to inventive cosmetic agents which, relative to their weight, contain from about 0.1 to about 5.0 wt. %, preferably from about 0.15 to about 2.5 wt. %, more preferably from about 0.175 to about 1.0 wt. %, even more preferably from about 0.2 to about 0.8 wt. % and particularly from about 0.25 to about 0.7 wt % of compound(s) from the group 1,2-hexanediol, 1,2-octanediol.

Particular preference is given to agents which contain, relative to their weight, from about 0.1 to about 2.0 wt. %, preferably from about 0.15 to about 1.5 wt. %, more preferably from about 0.2 to about 1.0 wt. %, even more preferably from about 0.25 to about 0.8 wt. % and particularly from about 0.3 to about 0.7 wt. % 1,2-hexanediol.

Preferably, the total sum of ingredients a) and b) is within a narrow range. In this context, cosmetic agents as contemplated herein are exemplified in that the total amount of ingredients a) and b), relative to the weight of the agent, is from about 0.25 to about 2.5 wt. %, preferably from about 0.4 to about 2 wt. %, more preferably from about 0.5 to about 1.5 wt. %, even more preferably from about 0.6 to about 1.0 wt. % and particularly from about 0.7 to about 0.9 wt. %.

The inventive cosmetic agents can contain additional deodorizing and/or perspiration-inhibiting substances. Preferred inventive cosmetic agents are exemplified in that they contain, in addition to one or multiple active ingredients, selected from the group of triethyl citrate, 3-[(2-ethylhexyl)oxy]-1,2-propanediol, 2-benzyl heptanol, phenoxyethanol, cocamidopropyl PG-dimonium chloride phosphate, tropolone, silver lactate, magnesium oxide, aluminum chlorohydrate, aluminum zirconium tetrachlorohydrex glycine and mixtures thereof.

The inventive cosmetic agents can contain certain aluminum salts. Therefore, it is preferable in the scope of the present disclosure if the cosmetic agent contains at least one aluminum salt selected from the group of (i) aqueous inorganic salts of aluminum, particularly aluminum chlorohydrate, aluminum sesquichlorohydrate, aluminum dichlorohydrate, aluminum hydroxide, potassium aluminum sulphate, aluminum bromohydrate, aluminum chloride, aluminum sulphate; (ii) water-soluble organic salts of aluminum, in particular aluminum chlorohydrex-propylene glycol, aluminum chlorohydrex-polyethylene glycol, aluminum-propylene glycol complexes, aluminum sesquichlorohydrex-propylene glycol, aluminum sesquichlorohydrex-polyethylene glycol, aluminum-propylene glycol-dichlorohydrex, aluminum-polyethylene glycol-dichlorohydrex, aluminum undecylenoyl collagen amino acid, sodium aluminum lactate, sodium aluminum chlorohydroxylactate, aluminum lipoamino acids, aluminum lactate, aluminum chlorohydroxyallantoinate, sodium aluminum chlorohydroxylactate; and (iii) mixtures thereof.

Furthermore, the inventive cosmetic agents can contain special aluminum zirconium salts. Therefore, further embodiments are exemplified in that the cosmetic agent contains at least one aluminum zirconium salt, selected from the group of (i) water-soluble inorganic aluminum zirconium salts, in particular aluminum zirconium trichlorohydrate, aluminum zirconium tetrachlorohydrate; aluminum zirconium tetrachlorohydrate; aluminum zirconium pentahydrate; aluminum zirconium octahydrate; (ii) water-soluble organic aluminum zirconium salts, in particular aluminum zirconium-propylene glycol complexes, aluminum zirconium tetrachlorohydrex glycine, aluminum zirconium tetrachlorohydrex glycine, aluminum zirconium tetrachlorohydrex glycine, aluminum zirconium octachlorohydrex glycine; and (iii) mixtures thereof.

As contemplated herein, the terms “aluminum salts” and “aluminum zirconium salts” are not understood to include any aluminosilicates and zeolites. Furthermore, in the context of the present disclosure, water-soluble aluminum salts and/or water-soluble aluminum zirconium salts are understood to mean salts having a solubility of at least 3 wt. % at 20° C., which means at least 3 g of the antiperspirant aluminum salt and/or aluminum zirconium salt dissolves in 97 g of water at 20° C.

Particularly preferred inorganic aluminum salts are selected from aluminum chlorohydrate, particularly aluminum chlorohydrate with the general formula [Al2(OH)5Cl 1-6 H2O]n, preferably [Al2(OH)5Cl.2-3 H2O]n, which can be in non-activated (polymerized) or in activated form (depolymerized), as well as aluminum chlorohydrate with the general formula [Al2(OH)4Cl2.1-6 H2O]n, preferably [Al2(OH)4Cl2.2-3 H2O]n, which can be in non-activated (polymerized) or in activated form (depolymerized).

As contemplated herein, particularly preferred antiperspirant aluminum salts are selected from so-called “activated” aluminum salts, which are also designated as antiperspirant active ingredients “with enhanced activity”. Activated aluminum salts are normally generated by employing heat treatment of a thin solution of the respective sale (e.g. a solution with 10 wt. % salt) in order to enlarge its HPLC-peak 4-to-peak 3 surface ratio. Then the activated salt can be dried to a powder, particularly spray-dried. In addition to the spray-drying, roll drying, for example, is also suitable. Activated aluminum salts typically have an HPLC-peak 4-to-peak 3 surface ratio of at least 0.4, preferably of at least 0.7, particularly of at least 0.9, wherein at least 70% of the aluminum are associated with these HPLC-peaks.

In this context, “activated” aluminum zirconium salts are also known, which have a high HPLC-peak 5-aluminum content, particularly a peak 5 surface of at least 33%, preferably of at least 45%, relative to the total surface under peaks from about 2 to about 5, measured with HPLC of a 10 wt. % aqueous solution of the active ingredient under conditions in which the aluminum species are dissolved in at least 4 peaks in succession (identified as peaks 2 to 5). Preferred aluminum zirconium salts with a high HPLC-peak 5-aluminum content is also designated as “ESAZCH”. The aforementioned activated aluminum zirconium salt can also be stabilized with a water-soluble strontium salt and/or with a water-soluble calcium salt.

As contemplated herein, it is also possible to use antiperspirant aluminum salts as anhydrous solutions or solubilizates of an activated antiperspirant aluminum or aluminum zirconium salt. Such aluminum salts or aluminum zirconium salts are stabilized against the loss of activation of the salt with the addition of an effective amount of a polyhydric alcohol, which contains 3 to 6 carbon atoms and 3 to 6 hydroxyl groups, preferably propylene glycol, sorbitol and pentaerythritol.

Particular preference is also given to complexes of activated antiperspirant aluminum salts or aluminum zirconium salts with a polyhydric alcohol, which contains from about 20 to about 50 wt. %, preferably from about 20 to about 42 wt. %, activated antiperspirant aluminum salt or aluminum zirconium salt and from about 2 to about 16 wt. % molecularly bound water, wherein 100 wt. % of the remainder is at least one polyhydric alcohol having 3 to 6 carbon atoms and 3 to 6 hydroxyl groups. Propylene glycol, propylene glycol/sorbitol mixtures and propylene glycol/pentaerythritol mixtures are preferred alcohols in this context.

In the context of the present disclosure, it is also possible to use basic calcium aluminum salts as aluminum salts. These salts can be obtained by reacting calcium carbonate with aluminum chlorohydroxide or aluminum chloride and aluminum powder or by adding calcium chloride dihydrate to aluminum chlorohydroxide. However, it is also possible to use aluminum zirconium complexes which are buffered with salts of amino acids, in particular with alkali and alkaline earth glycinates.

Aluminum salts and aluminum zirconium salts which are preferably stabilized by amino acids, particularly glycerin, hydroxyalkanoic acids, in particular glycolic acid and lactic acid, or betaines, can also be used as preferred antiperspirant aluminum salts and aluminum zirconium salts as contemplated herein.

Additional preferred activated aluminum salts have the general formula Al2(OH)6-aXa, wherein X denotes Cl, Br, I or NO3 and “a” denotes a number from about 0.3 to about 5, preferably from about 0.8 to about 2.5, particularly from about 1 to about 2, so that the mol ratio is from Al:X from about 0.9:1 to about 2.1:1. Aluminum chlorohydrate (which means X denotes Cl in the aforementioned formula) is preferred and especially 5/6 basic aluminum chlorohydrate with “a”=1, so that the mol ratio of aluminum to chlorine is from about 1.9:1 to about 2.1:1.

Preferred activated aluminum zirconium salts have the general formula ZrO(OH)2-pbYb, wherein Y denotes Cl, Br, I, NO3 or SO4, b denotes a rational number from 0.8 to 2 and p denotes the valence of Y, so that the Al:Zr mol ratio is 2 to 10 and the metal:(X+Y) ratio is from about 0.73 to about 2.1, preferably from about 0.9 to about 1.5. A particularly preferred salt is aluminum zirconium chlorohydrate (which means X and Y stand for CO, which has an Al:Zr ratio of from about 2 to about 10 and a molar metal:Cl ratio of from about 0.9 to about 2.1.

As contemplated herein, particularly preferred antiperspirant aluminum salts have a molar metal-to-chloride ratio of from about 1.9 to about 2.1. The metal-to-chloride ratio of particularly preferred aluminum sesquichlorohydrates in the context of the present disclosure is from about 1.5:1 to about 1.8:1. Preferred aluminum zirconium tetrachlorohydrates have a molar ratio of Al:Zr of 2 to 6 and of metal:chloride of from about 0.9 to about 1.3, wherein particular preference is given to salts with a molar metal-to-chloride ratio of from about 0.9 to about 1.1, preferably from about 0.9 to about 1.0.

In the context of the present disclosure, preference is given use specific active ingredients mentioned above. Preferred embodiments of the present disclosure are therefore exemplified in that the cosmetic agent contains at least one first active ingredient, selected from the group of triethyl citrate, 3-[(2-ethylhexyl)oxy]-1,2-propanediol, 2-benzyl heptanol, phenoxyethanol, cocamidopropyl PG-dimonium chloride phosphate, tropolone, silver lactate, magnesium oxide, aluminum chlorohydrate, aluminum zirconium tetrachlorohydrex glycine and mixtures thereof. Use of these active ingredients has been shown to be particularly beneficial in regard to the deodorizing and/or antiperspirant effect. In particular, a synergistic improvement of the deodorizing and/or antiperspirant effect is achieved. Moreover, use of these active ingredients further improves the skin compatibility and the cosmetic properties of the inventive agents. These compounds have the following chemical names and CAS numbers:

| Preservative | Chemical name/structure formula | CAS number |
|---|---|---|
| Triethyl citrate | 2-hydroxypropane-1,2,3-tricarboxylic acid | 77-93-0 |
| | triethyl ester | |
| 3-[(2-ethylhexyl)oxy]-1,2- | OCTOXYGLYCERIN | 70445-33-9 |
| propanediol | | |
| 2-benzylheptanol | 2-benzyl-1-heptanol | 92368-90-6 |
| Phenoxyethanol | 2-phenoxy-1-ethanol | 122-99-6 |
| Cocamidopropyl | Propanaminum, 2,3-dihydroxy-N,N- | 83682-78-4 |
| PG-dimonium chlorophosphate | dimethyl-N-(coconut-alkyl)-, 3-sodium | |
| | hydrogen phosphate ester, chloride | |
| Tropolone | 2-hydroxy-2,4,6-cycloheptatriene-1-one | 38768-08-0 |
| Silver lactate | Silver lactate | 128-00-7 |
| Magnesium oxide | Magnesium oxide, MgO | 1309-48-4 |
| Aluminum chlorohydrate | Aluminum chloride, basic | 1327-41-9 |
| Aluminum zirconium | Al₄Zr(OH)₁₂Cl₄Gly × nH₂O | 90604-80-1 |
| tetrachlorohydrex glycine | | |

The deodorizing and/or antiperspirant effect can be further improved if the cosmetic agents contain more than one of the active ingredients listed above. Therefore, preferred cosmetic agents are exemplified in that they contain at least two active ingredients selected from the group of triethylcitrate, 3-[(2-ethylhexyl)oxy]-1,2-propanediol, 2-benzylheptanol, phenoxyethanol, cocamidopropyl PG-dimoniumchloridphosphate, 1,2-hexanediol, 1,2-octanediol, 1,2-decanediol, tropolone, silver lactate, magnesium oxide, aluminum chlorohydrate, aluminum zirconium tetrachlorohydrex glycine and mixtures thereof.

Additional preferred cosmetic agents as contemplated herein are exemplified in that they contain at least three active ingredients selected from the group of triethylcitrate, 3-[(2-ethylhexyl)oxy]-1,2-propanediol, 2-benzylheptanol, phenoxyethanol, cocamidopropyl PG-dimonium chlorophosphate, 1,2-hexanediol, 1,2-octanediol, 1,2-decanediol, tropolone, silver lactate, magnesium oxide, aluminum chlorohydrate, aluminum zirconium tetrachlorohydrex glycine and mixtures thereof.

The inventive cosmetic agents preferably contain the aforementioned active ingredient(s) in specific quantity ranges. Therefore, preferred cosmetic agents as contemplated herein are exemplified in that they contain from about 0.0005 to about 35 wt. %, preferably from about 0.005 to about 25 wt. %, more preferably from about 0.01 to about 10 wt. %, particularly from about 0.05 to about 8.0 wt. % of at least one active ingredient selected from the group of triethylcitrate, 3-[(2-ethylhexyl)oxy]-1,2-propanediol, 2-benzylheptanol, phenoxyethanol, cocamidopropyl PG-dimonium chlorophosphate, 1,2-hexanediol, 1,2-octanediol, 1,2-decanediol, tropolone, silver lactate, magnesium oxide, aluminum chlorohydrate, aluminum zirconium tetrachlorohydrex glycine and mixtures thereof. Use of such quantities of the at least one active ingredient achieves a synergistic improvement of the deodorizing and/or antiperspirant effect. As a result the amount of active ingredients used can be reduced without negatively influencing the deodorizing and/or antiperspirant effect. Therefore, the inventive cosmetic agents are particularly skin-friendly.

With the powerful inventive combination, the inventive agents can also be formulated without using aluminum salts without reducing performance. Normally, the effectiveness against body odor of aluminum-free products is comparatively low, wherein the performance deficit is particularly noticeable after from about 24 to about 48 hours. A significant improvement of the deodorizing effect during the period from about 24 to about 48 hours after application is achieved with the inventive combination.

Preferred cosmetic agents as contemplated herein are exemplified in that they are free from aluminum salts.

As an additional component, the inventive cosmetic agents can contain at least one active ingredient selected from the group of cetylpyridinium chloride, benzalkonium chloride, benzethonium chloride, zinc hydroxide carbonate, zinc phenolsulfonate, polyglyceryl-2-caprate, sorbitan caprylate, octenidine, charnesolic acid, tartaric acid and mixtures thereof. Preferred cosmetic agents in the context of the present disclosure are exemplified in that they also contain one or multiple active ingredients selected from the group of cetylpyridinium chloride, benzalkonium chloride, benzethonium chloride, zinc hydroxide carbonate, zinc phenolsulfonate, polyglyceryl-2-caprate, sorbitan caprylate, octenidine, charnesolic acid, tartaric acid and mixtures thereof.

In the context of the present disclosure, preference is given use of certain active ingredients. Therefore, preferred embodiments of the present disclosure are exemplified in that the cosmetic agent contains at least one additional active ingredient selected from the group of cetylpyridinium chloride, benzalkonium chloride, benzethonium chloride, zinc hydroxide carbonate, zinc phenolsulfonate, polyglyceryl-2-caprate, sorbitan caprylate, octenidine, charnesolic acid, tartaric acid and mixtures thereof.

These compounds have the following chemical names and CAS numbers:

| Preservative | Chemical name/structure formula | CAS number |
|---|---|---|
| Cetylpyridinium chloride | 1-hexadecylpyridinium chloride | 123-03-5 |
| Benzalkonium chloride | N-C₈-C₁₈-alkyl-N-benzyl-N,N- | 8001-54-5 |
| | dimethylammonium chloride | |
| Benzethonium chloride | Diisobutylphenoxy ethoxyethyldimethyl | 121-54-0 |
| | benzylammonium chloride | |
| Zinc hydroxide carbonate | [ZnCO₃]₂*[Zn(OH)₂]₃ | 5263-02-5 |
| Zinc phenolsulfonate | Zinc phenolsulfonate | 127-82-2 |
| Polyglyceryl-2-caprate | 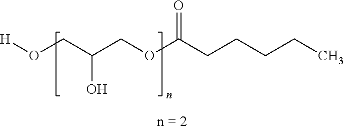 | 156153-06-9 |
| Sorbitan caprylate | Sorbitan monooctanoate | 60177-36-8 |
| Octenidine | N-Octyl-1-[10-(4-octyliminopyridine-1- | 70775-75-6 |
| | yl)decyl]pyridine-4-imin dihydrochloride | |
| Charnesolic acid | (4aR,10aS)-5,6-dihydroxy-1,1-dimethyl-7- | 3650-09-7 |
| | propan-2-yl-2,3,4,9,10,10a- | |
| | hexahydrophenanthrene-4a-carboxylic acid | |
| Tartaric acid | D- and/or L-tartaric acid | 133-37-9 |

The deodorizing and/or antiperspirant effect can be further improved if the cosmetic agents contain more than one of the active ingredients listed above. Therefore, preferred cosmetic agents are exemplified in that they contain at least two active ingredients selected from the group of cetylpyridinium chloride, benzalkonium chloride, benzethonium chloride, zinc hydroxide carbonate, zinc phenolsulfonate, polyglyceryl-2-caprate, sorbitan caprylate, octenidine, charnesolic acid, tartaric acid and mixtures thereof.

Additional referred cosmetic agents in the context of the present disclosure are exemplified in that they contain at least three active ingredients selected from the group of etylpyridinium chloride, benzalkonium chloride, benzethonium chloride, zinc hydroxide carbonate, zinc phenolsulfonate, polyglyceryl-2-caprate, sorbitan caprylate, octenidine, charnesolic acid, tartaric acid and mixtures thereof.

The inventive cosmetic agents preferably contain the second active ingredient in specific quantity ranges. Therefore, preferred cosmetic agents in the context of the present disclosure are exemplified in that they contain, relative to their total weight, from about 0.001 to about 20 wt. %, preferably from about 0.005 to about 15 wt. %, more preferably from about 0.01 to about 10 wt. %, particularly from about 0.05 to about 2.0 wt. % of at least one additional active ingredient selected from the group of cetylpyridinium chloride, benzalkonium chloride, benzethonium chloride, zinc hydroxide carbonate, zinc phenolsulfonate, polyglyceryl-2-caprate, sorbitan caprylate, octenidine, charnesolic acid, tartaric acid and mixtures thereof.

The inventive cosmetic agents can also contain additional deodorant active ingredients (C). However, the additional deodorant active ingredient (C) is different from the first active ingredient (A) and the second active ingredient (B).

In the context of the present disclosure, it can be preferable if the cosmetic agent also contains a deodorant active ingredient (C). Therefore, preferred agents are exemplified in that they contain at least one deodorant active ingredient (C) selected from the group of (i) active ingredients against exoesterases, in particular against arylsulfatase, lipase, beta-glucuronidase and cystathionine β-lyase; (ii) odor absorbers, in particular silicates, such as montmorillonite, kaolinite, illite, beidellite, nontronite, saponite, hectorite, bentonite, smectite and talcum, zeolites, zinc ricinoleate, cyclodextrins; (iii) deodorizing ion exchangers; (iv) germ inhibiting agents; (v) prebiotically effective components and (vi) mixtures thereof.

Silicates serve as odor absorbers which can simultaneously support the rheological properties of the inventive cosmetic agents. Particularly beneficial silicates as contemplated herein include, above all, sheet silicates, and, among these, in particular montmorillonite, kaolinite, illite, beidellite, nontronite, saponite, hectorite, bentonite, smectite and talcum. Additional examples of beneficial odor absorbers are zeolites, zinc ricinoleate, cyclodextrins, certain metal oxides, such as aluminum oxide, as well as chlorophyll.

Furthermore, the deodorant active ingredient can be selected from the group of terpene alcohols such as farnesol, chlorophyllin-copper complexes, carboxylic acid esters, in particular carboxylic acid monoesters of mono-, di- and triglycerol (in particular glycerol monolaurate, diglycerol monocaprinate, diglycerol monolaurate, triglycerol monolaurate and triglycerol monomyristate) and plant extracts (e.g. green tee and components of lime blossom oil).

Other preferred deodorant active ingredients are selected from prebiotic active components, which, in the context of the present disclosure, are understood to mean only or at least predominantly inhibit the odor-forming germs in the skin microflora, but not the desirable, i.e. not odor-forming, germs. This explicitly includes conifer extracts, in particular from the group of pinaceae, and plant extracts from the group of sapindaceae, araliaceae, lamiaceae and sxifragaceae, in particular extracts from picea spp., paullinia sp, panax sp., lamium album or ribes nigrum, and mixtures of these substances.

Further preferred deodorant active ingredients are selected from the germ-inhibiting perfume oils and Deosafe perfume oils, which are available from Symrise (formerly Haarmann and Reimer).

The enzyme inhibitors include substances which inhibit the enzymes responsible for decomposition of perspiration, particularly arylsulfatase, beta-glucuronidase, aminoacylase, ester-cleaving lipases and lipoxygenases, such as zinc glycinate.

The at least one additional deodorant active ingredient (C) is preferably used in the inventive cosmetic agents in specific quantity ranges. Therefore, in this context is preferable that they contain—relative to their total weight—from about 0.005 to about 20 wt. %, preferably from about 0.1 to about 15 wt. %, more preferably from about 0.1 to about 13 wt. %, particularly from about 0.1 to about 10 wt. % of at least one deodorant active ingredient (C). If a mixture of deodorant active ingredients is used, the aforementioned quantity specifications relate to the mixture of said active ingredients.

In the scope of the present disclosure it can be stipulated that the inventive cosmetic agent contains, in addition to the additional deodorant ingredients indicated above, additional ingredients selected from the group of (i) waxes, (ii) emulsifiers and/or surfactants; (iii) hydrogel formers; (iv) skin-cooling active ingredients; (v) propellants; (vi) thickening agents and (vii) mixtures thereof.

The inventive cosmetic agents can also contain at least one wax. The term “waxes” in the scope of the present disclosure is understood to mean substances which are kneadable or firm to stiff and brittle at 20° C., have a rough to fine-crystalline structure and are transparent to opaque in color, but not glassy. These substances also melt at temperatures above 25° C. without breaking down, are only slightly fluid above the melting point (less viscous), have a consistent and solubility that are highly temperature-independent and are polishable under light pressure. Therefore, as contemplated herein it is advantageous if the cosmetic agent also contains a wax selected from the group of (i) coconut fatty acid glycerol mono-, -di-and-triesters; (ii) butyrospermum parkii (shea butter); (iii) esters of saturated, monohydric C₈₋₁₈-alcohols with saturated C₁₂₋₁₈-monocarboxylic acids; (iv) linear, primary C₁₂-C₂₄-alkanols; (v) esters of a saturated, monohydric C₁₆-C₆₀-alkanol and a saturated C₈-C₃₆-monocarboxylic acid, in particular cetyl behenate, stearyl behenate and C₂₀-C₄₀-alkyl stearate; (vi) glycerol triesters of saturated linear C₁₂-C₃₀-carboxylic acids which can be hydroxylated, in particular hydrogenated palm oil, hydrogenated coconut oil, hydrogenated castor oil, glyceryl tribehenate and glyceryl tri-12-hydroxystearate; (vii) natural vegetable waxes, in particular candelilla wax, carnauba wax, Japan wax, sugar cane wax, ouricury wax, cork wax, sunflower wax, fruit waxes; (viii) animal waxes, in particular beeswax, shellac wax and spermaceti; (ix) synthetic waxes, in particular montan ester waxes, hydrogenated jojoba waxes and sasol waxes, polyalkylene waxes and polyethylene glycol waxes, C₂₀-C₄₀-dialkyl esters of dimer acids, C₃₀₋₅₀-alkyl beeswax and alkyl and alkylaryl esters of dimer fatty acids, paraffin waxes; and (x) mixtures thereof. The cosmetic agent also preferably contains the at least one wax in a total quantity of from about 0.01 to about 20 wt. %, preferably from about 3 to about 20 wt. %, more preferably from about 5 to about 18 wt. %, particularly from about 6 to about 15 wt. %, relative to the total weight of the cosmetic agent.

As contemplated herein, suitable emulsifiers and surfactants are preferably selected from anionic, cationic, non-ionic, amphoteric, particularly ampholytic and zwitterionic emulsifiers and surfactants. Surfactants are amphiphilic (bifunctional) compounds that include of at least one hydrophobe and at least one hydrophile molecular part. The hydrophobic radical is preferably a hydrocarbon chain with 8 to 28 carbon atoms, which can be saturated or unsaturated, linear or branched. It is especially preferable if this C₈-C₂₈ alkyl chain is linear.

Anionic surfactants are understood to mean surfactants having exclusively anionic charges; they contain, for example, carboxyl groups, sulfonic acid groups or sulfate groups. Particularly preferred anionic surfactants are alkyl sulfates, alkyl ether sulfates, acylglutamates and C₈₋₂₄-carboxylic acids and salts thereof, so-called soaps.

Cationic surfactants are understood to mean surfactants having exclusively cationic charges; they contain, for example, quaternary ammonium groups. Preference is given to cationic surfactants of the type of quaternary ammonium compounds, esterquats and amidoamines. Preferred quaternary ammonium compounds are ammonium halides and the imidazolium compounds known under the INCI designations quaternium-27 and quaternium-83. As contemplated herein, the quaternized protein hydrolysates can also be used. Preferred esterquats are quaternated ester salts of fatty acids with triethanolamine, quaternated ester salts of fatty acids with diethanolalkyl amines and quaternated ester salts of fatty acids with 1,2-dihydroxypropyldialkylamines.

The amphoteric surfactants are divided into ampholytic surfactants and zwitterionic surfactants. Ampholytic surfactants are surface active compounds that contain both acid (for example, —COOH or —SO₃H groups) and also alkaline hydrophilic groups (for example, amino groups) and have acidic or alkaline behavior depending on the condition. Zwitterionic surfactants are specialist surfactants that carry both a negative and a positive charge in the same molecule. Examples of preferred zwitterionic surfactants are betaines, the N-alkyl-N, N-dimethylammoniumglycinates, N-acylaminopropyl-N, N-dimethylammoniumglycinate and the 2-alkyl-3-carboxymethyl-3-hydroxyethyl-imidazolines, each having 8 to 24 carbon atoms in the alkyl group. Examples of the preferred ampholytic surfactants are n-alkylglycines, n-alkylaminopropionic acids, n-alkylaminobutyric acids, n-alkyliminodipropionic acids, n-hydroxyethyl-n-alkylamidoamidopropyl-glycines, n-alkyl taurines, n-alkyl sarcosines, 2-alkylaminopropionic acids and alkylaminoacetic acids having in each case from about 8 to about 24 carbon atoms in the alkyl group.

The inventive cosmetic agents, which are formulated as an emulsion, particularly an oil-in-water emulsion, preferably contain at least one nonionic oil-in-water emulsifier with an HLB value of more than about 7 to about 20. These are emulsifiers that are generally known to a person skilled in the art. The formula HLB=(100−L): 5 is calculated for ethoxylated products, wherein L denotes weight ratio of the lipophilic groups, i.e. fatty alkyl or fatty acyl groups in which ethylene oxideadducts, expressed in percent by weight. In this context, use of an additional water-in-oil emulsifier with an HLB value greater than 1.0 and less than or equal to 7.0 can be given preference as contemplated herein.

As contemplated herein, for the thickening of the inventive cosmetic agents, preference is given to the use of hydrogel-forming substances selected from cellulose ethers, particularly hydroxyalkyl celluloses, especially hydroxypropyl cellulose, hydroxypropyl methyl cellulose, hydroxyethyl cellulose, carboxymethyl cellulose, cetyl hydroxyethyl cellulose, hydroxybutylmethylcellulose, methylhydroxyethylcellulose, as well as xanthan gum, sclerotium gum, succinoglucans, polygalactomannan, particularly guar gum and locust bean gum, particularly guar gum and locust bean gum itself and the nonionic hydroxyalkyl guar derivatives and locust bean gum derivatives, such as hydroxypropyl guar, carboxymethyl hydroxypropyl guar, hydroxypropyl methyl guar, hydroxyethyl guar and carboxymethyl guar, also pectins, agar, carragheen (carrageenan), tragacanth, gum arabic, karaya gum, tara gum, gellan, gelatin, casein, propylene glycol alginate, alginic acids and the salts thereof, in particular sodium alginate, potassium alginate and calcium alginate, as well as polyvinylpyrrolidones, polyvinyl alcohols and polyacrylamides. Particularly preferred hydrogel formers are selected from cellulose ethers, particularly hydroxyalkyl celluloses, especially hydroxypropyl cellulose, hydroxypropyl methyl cellulose, hydroxyethyl cellulose, carboxymethyl cellulose, cetyl hydroxyethyl cellulose, hydroxybutylmethylcellulose, methylhydroxyethylcellulose and mixtures thereof. However, a lipophilic thickening agent can also be used. Preferred lipophilic thickening agents as contemplated herein are selected from hydrophized clay minerals and pyrogenic silicas.

The inventive cosmetic agents can also contain at least one skin-cooling active ingredient. Suitable skin-cooling active ingredients as contemplated herein are, for example, menthol, isopulegol and menthol derivatives, such as menthyl lactate, menthyl glycolate, menthyl ethyl oxamates, menthyl pyrrolidone carboxylic acid, menthyl methyl ether, menthoxypropane diol, menthoxyglycerol acetal (9-methyl-6-(1-methylethyl)-1,4-dioxaspiro (4.5) decane-2-methanol), monomenthyl succinate, 2-hydroxymethyl-3,5,5-trimethylcyclohexanol and 5-methyl-2-(1-methylethyl) cyclohexyl-n-ethyl oxamate. Menthol, isopulegol, menthyl lactate, menthoxypropane diol, menthyl pyrrolidone carboxylic acid and 5-methyl-2-(1-methylethyl)cyclohexyl-n-ethyl oxamate and mixtures of these substances, in particular mixtures of menthol and menthyl lactate, menthol, menthol glycolate and menthyl lactate, menthol and menthoxypropanediol or menthol and isopulegol, are preferred as skin-cooling active ingredients.

Furthermore, it can be stipulated that the inventive cosmetic agents contain a propellant. In this case, they are packaged as propellant-gas-operated aerosols. Preferred propellants (propellant gases) are propane, propene, n-butane, iso-butane, iso-butene, n-pentane, pentene, iso-pentane, iso-pentene, methane, ethane, dimethylester, nitrogen, air, oxygen, nitrous oxide, 1,1,1,3-tetrafluorethane, heptafluoro-n-propane, perfluoroethane, monochlorodifluormethane, 1,1-difluorethane and tetrafluoropropenes, both individually and in mixtures thereof. Hydrophilic propellant gases, such as carbon dioxide, can also be used advantageously in the context of the present disclosure if the proportion of hydrophilic gases is selected to be low and lipophilic propellant gas (such as propane/butane) is present in excess. Propane, n-butane, iso-butane and mixtures of these propellant gases are especially preferred. It has been found that the use of n-butane as the only propellant gas can be particularly preferred as contemplated herein. The total amount of propellant is from about 20 to about 95 wt. %, preferably from about 30 to about 85 wt. %, particularly from about 40 to about 75 wt. %, relative to the total weight of the cosmetic agent in each case, including of the components a) and b) described above, as well as the additional active ingredients and ingredients and the propellant, if applicable.

Application of the cosmetic agent as contemplated herein can take place by employing different methods. According to a first embodiment, the cosmetic agent is packaged as a spray application. The spray application is carried out with a spray device containing a filling in a container of the liquid, viscous flowable, suspension-like or powdery antiperspirant cosmetic agent. The filling can be carried out under the pressure of a propellant (compressed gas cans, compressed gas packs, aerosol packs), or it can be a pump atomizer without propellant gas (pump spray/squeeze bottle) to be operated mechanically. The containers have a removal device, preferably in the form of valves, which enable removal of the contents as a mist, smoke, foam, powder, paste or liquid jet. Cylindrical containers made of metal (aluminum, tinplate, maximum capacity preferably 1,000 ml), protected and/or shatter-proof glass or plastic (maximum capacity preferably 220 ml) or shattering glass or plastic (maximum capacity preferably from about 50 to about 400 ml) is a suitable example of a container for such cosmetic products. Creme-like, gelatinous, pasty and liquid agents can be packaged, for example, in the form of pump, spray or squeeze dispensers, more particularly also in the form of multi-chamber pump, multi-chamber spray or multi-chamber squeeze dispensers. Packing for the inventive agent can be opaque, transparent or translucent.

According to a second embodiment, the inventive cosmetic agent can be packaged as a stick. soft solid, cream, roll-on, dibenzylidenalditol-based gel or loose or compact powder. The formulation of the inventive cosmetic agents in a specific dosage form, such as an antiperspirant roll-on, an antiperspirant stick or an antiperspirant gel is preferentially based on the requirements of the intended use. Therefore, depending on the intended use, the cosmetic agents as contemplated herein can exist in solid, semi-solid, liquid, dispersed, emulsified, suspended, gelatinous, multi-phase or powder form. For the purposes of the present disclosure, the term “liquid” also encompasses any types of solid-state dispersions in liquids. Moreover, multi-phase cosmetic agents as contemplated herein are agents which have at least 2 different phases with a phase separation, and wherein the phases can be arranged horizontally, i.e. one above the other, or vertically, i.e. next to one another.

Application can take place, for example with a roller ball applicator. Such rollers have a ball supported in a ball bed, which can be moved by moving over a surface. In the process, the ball collects some of the inventive cosmetic agent to be distributed and applies it to the surface to be treated. The packing for these agents can be opaque, transparent or translucent, as described above.

Furthermore, it is also possible to apply the cosmetic agents as contemplated herein by employing a solid stick on the firm of a fixed emulsion.

According to a third embodiment, the inventive cosmetic agent can be selected from the group of wipes, pads and dabbers. Particular preference is given to wet wipes, in other words wet wipes prefabricated for the user, preferably packaged individually, which, for example, are well known from the field of glass cleaning or from the field of moist toilet papers. Such wet wipes, which can advantageously also contain preservatives, are coated with a cosmetic agent as contemplated herein and are preferably individually packaged. They can be used, for example, as a deodorant wipe, which is particularly relevant for use while traveling. Preferable substrate materials are selected from porous flat cloths. They can consist of a fibrous or cellular, flexible material which has adequate mechanical stability and, at the same time, softness for use on the skin. These cloths include cloths made of woven and non-woven (fleece) synthetic and natural fibers, felt, paper or foam, such as hydrophilic polyurethane foam. Preferred deodorizing or antiperspirant substrates as contemplated herein can be produced by saturation or impregnation or also by melting an inventive cosmetic agent are obtained on a substrate.

A second subject of the present disclosure is a non-therapeutic cosmetic method for preventing and/or reducing the body odor released by perspiration and/or perspiration of the body, wherein an inventive cosmetic agent is applied to the skin, particularly to the skin of the armpits, and remains on the skin for at least about 1 hour, preferably at least about 2 hours, more preferably at least about 4 hours, particularly for at least about 6 hours.

With respect to additional preferred embodiments of the inventive method, particularly with regard to the cosmetic agents use there, the statements made about the cosmetic agents as contemplated herein apply mutatis mutandis.

Finally, a further subject of the present disclosure is use of the inventive cosmetic agent to prevent and/or reduce the body odor released by perspiration.

With respect to other preferred embodiments of use as contemplated herein, more particularly with respect to the used agent, the statements made about the cosmetic agent as contemplated herein and also about the cosmetic product as contemplated herein apply mutatis mutandis.

### Examples

1. Deodorant Roll-on with Alcohol

| | 1A | 1B | 1C | 1D |
|---|---|---|---|---|
| | wt. % | wt. % | wt. % | wt. % |
| Hydroxyethylcellulose | 0.4 | 0.4 | 0.4 | 0.4 |
| 2-butyloctanoic acid | 0.3 | 0.5 | 0.7 | 0.4 |
| 1,2-hexanediol | 0.4 | 0.2 | 0.1 | 0.5 |
| Phenoxyethanol | 0.5 | 0.5 | 0.5 | 0.5 |
| Triethyl citrate | 1.0 | 1.0 | 1.0 | 1.0 |
| Ethanol 96% | 30.0 | 30.0 | 30.0 | 30.0 |
| Ceteareth-12 | 2.0 | 2.0 | 2.0 | 2.0 |
| Ceteareth-30 | 2.0 | 2.0 | 2.0 | 2.0 |
| Perfume | 1.0 | 1.0 | 1.0 | 1.0 |
| Water | Ad 100 | Ad 100 | Ad 100 | Ad 100 |

2. Deodorant Roll-on without Alcohol

| | 2A | 2B | 2C | 2D |
|---|---|---|---|---|
| | wt. % | wt. % | wt. % | wt. % |
| PPG-15 Stearyl ether | 0.5 | 0.5 | 0.5 | 0.5 |
| Steareth-2 | 2.4 | 2.4 | 2.4 | 2.4 |
| Steareth-21 | 1.5 | 1.5 | 1.5 | 1.5 |
| 2-butyloctanoic acid | 0.3 | 0.5 | 0.7 | 0.4 |
| 1,2-octanediol | 0.4 | 0.2 | 0.1 | 0.5 |
| Phenoxyethanol | 0.5 | 0.5 | 0.5 | 0.5 |
| EDETA BX Powder | 0.1 | 0.1 | 0.1 | 0.1 |
| Perfume | 1.0 | 1.0 | 1.0 | 1.0 |
| Water | Ad 100 | Ad 100 | Ad 100 | Ad 100 |

### 3. Deodorant Atomizer

| | 3A | 3B | 3C | 3D |
|---|---|---|---|---|
| | wt. % | wt. % | wt. % | wt. % |
| PEG-40 Hydrogenated Castor Oil | 0.5 | 0.5 | 0.5 | 0.5 |
| 2-butyloctanoic acid | 0.3 | 0.5 | 0.7 | 0.4 |
| 1,2-hexanediol | 0.4 | 0.2 | 0.1 | 0.5 |
| Triethyl citrate | 3.0 | 3.0 | 3.0 | 3.0 |
| Ethanol 96% | 55.0 | 55.0 | 55.0 | 55.0 |
| Perfume | 1.0 | 1.0 | 1.0 | 1.0 |
| Water | Ad 100 | Ad 100 | Ad 100 | Ad 100 |

### 4. Deodorant Aerosol

| | 4-1 | 4-2 | 4-3 | 4-4 | 4-5 | 4-6 | 4-7 | 4-8 |
|---|---|---|---|---|---|---|---|---|
| Ethanol 96% | ad 100 | | | | | | | |
| 2-butyloctanoic acid | 0.3 | 0.5 | 0.7 | 0.4 | 0.3 | 0.5 | 0.7 | 0.4 |
| 1,2-hexanediol | — | — | — | — | 0.3 | 0.1 | 0.5 | 0.2 |
| 1,2-octanediol | 0.4 | 0.2 | 0.1 | 0.5 | 0.3 | 0.5 | 0.1 | 0.4 |
| Triethyl citrate | 6.0 | 6.0 | 6.0 | 6.0 | 6.0 | 6.0 | 6.0 | 6.0 |
| Phenoxy ethanol | 0.8 | 0.8 | 0.8 | 0.8 | 0.8 | 0.8 | 0.8 | 0.8 |
| Perfume | 4.0 | 4.0 | 4.0 | 4.0 | 4.0 | 4.0 | 4.0 | 4.0 |

### 5. Deodorant Aerosol

| | 5-1 | 5-2 | 5-3 | 5-4 | 5-5 | 5-6 | 5-7 | 5-8 |
|---|---|---|---|---|---|---|---|---|
| Cyclo- | 60.0 | 60.0 | 60.0 | 60.0 | 60.0 | 60.0 | 60.0 | 60.0 |
| pentasiloxane | | | | | | | | |
| Ethylhexyl | | | | | | | | |
| palmitate | ad 100 | | | | | | | |
| Dimethicone | 10.0 | 10.0 | 10.0 | 10.0 | 10.0 | 10.0 | 10.0 | 10.0 |
| 5 cSt | | | | | | | | |
| 2-butyloctanoic | 0.3 | 0.2 | 0.7 | 1.0 | 0.3 | 0.2 | 0.7 | 1.0 |
| acid | | | | | | | | |
| 1,2-hexanediol | — | 0.1 | 0.2 | 0.3 | 0.4 | 0.5 | 0.6 | 0.7 |
| 1,2-octanediol | 0.7 | 0.5 | 0.4 | 0.3 | 0.2 | 0.1 | 0.1 | — |
| Triethyl citrate | 6.0 | 6.0 | 6.0 | 6.0 | 6.0 | 6.0 | 6.0 | 6.0 |
| Phenoxy- | 0.8 | 0.8 | 0.8 | 0.8 | 0.8 | 0.8 | 0.8 | 0.8 |
| ethanol | | | | | | | | |
| Perfume | 4.0 | 4.0 | 4.0 | 4.0 | 4.0 | 4.0 | 4.0 | 4.0 |

The recipes are specified in the weight ratio of 1:3 with the propellant propane-butane (15/85) dispensed in aerosol cans.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic agent comprising, in a cosmetically compatible carrier,
a) 2-butyloctanoic acid and
b) at least one diol of the formula (I) 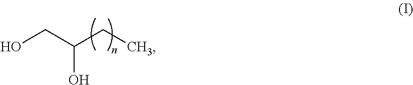
wherein n denotes an integer selected from the group of 3, 5, 7 and 9, wherein the total amount of compound(s) of the formula (I) is from about 0.05 to about 10 wt. %, relative to the total weight of the cosmetic agent.

2. Cosmetic agent according to claim 1, wherein the cosmetic agent comprises from about 0.01 to about 1 wt. %, 2-butyloctanoic acid, relative to the total weight of the cosmetic agent.

3. Cosmetic agent according to claim 1, wherein the cosmetic agent comprises at least one compound from the group 1,2-hexanediol, and 1,2-octanediol.

4. Cosmetic agent according to claim 1, wherein the cosmetic agent comprises from about 0.1 to about 5.0 wt. %, relative to the total weight of the cosmetic agent.

5. Cosmetic agent according to claim 1, wherein the cosmetic agent comprises from about 0.1 to about 2.0 wt. % 1,2-hexanediol, relative to the total weight of the cosmetic agent.

6. Cosmetic agent according to claim 1, wherein the total amount of ingredients a) and b), relative to the total weight of the cosmetic agent, is from about 0.25 to about 2.5 wt. %.

7. Cosmetic agent according to claim 1, wherein the cosmetic agent comprises one or multiple active ingredients selected from the group of triethyl citrate, 3-[(2-ethylhexyl)oxy]-1,2-propanediol, 2-benzyl heptanol, phenoxyethanol, cocamidopropyl PG-dimonium chloride phosphate, tropolone, silver lactate, magnesium oxide, aluminum chlorohydrate, aluminum zirconium tetrachlorohydrex glycine, and mixtures thereof.

8. Cosmetic agent according to claim 1, wherein the cosmetic agent further comprises one or multiple active ingredients selected from the group of cetylpyridinium chloride, benzalkonium chloride, benzethonium chloride, zinc hydroxide carbonate, zinc phenolsulfonate, polyglyceryl-2-caprate, sorbitan caprylate, octenidine, charnesolic acid, tartaric acid, and mixtures thereof.

9. Non-therapeutic cosmetic method for preventing and/or reducing body odor, the method comprising applying a cosmetic agent according to claim 1 to skin of the armpits, and allowing the cosmetic agent to remain on the skin for at least about 1 hour.

10. A method comprising using a cosmetic agent according to claim 1 to prevent and/or reduce body odor released by perspiration.

11. Cosmetic agent according to claim 1, wherein the cosmetic agent comprises from about 0.02 to about 0.8 wt. % 2-butyloctanoic acid, relative to the total weight of the cosmetic agent.

12. Cosmetic agent according to claim 1, wherein the cosmetic agent comprises from about 0.03 to about 0.7 wt. % 2-butyloctanoic acid, relative to the total weight of the cosmetic agent.

13. Cosmetic agent according to claim 1, wherein the cosmetic agent comprises from about 0.04 to about 0.6 wt. % 2-butyloctanoic acid, relative to the total weight of the cosmetic agent.

14. Cosmetic agent according to claim 1, wherein the cosmetic agent comprises from about 0.05 to about 0.5 wt. % 2-butyloctanoic acid, relative to the total weight of the cosmetic agent.

15. Cosmetic agent according to claim 1, wherein the cosmetic agent comprises from about 0.15 to about 2.5 wt. % of compound(s) from the group 1,2-hexanediol, and 1,2-octanediol, relative to the total weight of the cosmetic agent.

16. Cosmetic agent according to claim 1, wherein the cosmetic agent comprises from about 0.175 to about 1.0 wt. % of compound(s) from the group 1,2-hexanediol, and 1,2-octanediol, relative to the total weight of the cosmetic agent.

17. Cosmetic agent according to claim 1, wherein the cosmetic agent comprises from about 0.2 to about 0.8 wt. % of compound(s) from the group 1,2-hexanediol, and 1,2-octanediol, relative to the total weight of the cosmetic agent.

18. Cosmetic agent according to claim 1, wherein the cosmetic agent comprises from about 0.25 to about 0.7 wt % of compound(s) from the group 1,2-hexanediol, and 1,2-octanediol, relative to the total weight of the cosmetic agent.

19. Cosmetic agent according to claim 1, wherein the cosmetic agent comprises from about 0.15 to about 1.5 wt. % 1,2-hexanediol, relative to the total weight of the cosmetic agent.

20. Cosmetic agent according to claim 1, wherein the total amount of ingredients a) and b), relative to the total weight of the cosmetic agent, is from about 0.4 to about 2 wt. %.
